(19) United States
(12) Patent Application Publication
(10) Pub. No.: US 2021/0275703 A1
(43) Pub. Date: Sep. 9, 2021

(54) Self-Sterilizing Ultra-Violet Implement

(71) Applicant: Robert Joe Alderman, Poteet, TX (US)

(72) Inventor: Robert Joe Alderman, Poteet, TX (US)

(21) Appl. No.: 17/194,776

(22) Filed: Mar. 8, 2021

Related U.S. Application Data
(60) Provisional application No. 63/100,324, filed on Mar. 9, 2020.

(51) Int. Cl. A61L 2/10; A61L 2/24

(52) U.S. Cl. CPC A61L 2/10; A61L 2/24; A61L 2202/20; A61L 2202/121; A61L 2202/11

(57) ABSTRACT

A self-sterilizing implement with a manual contact surface and an ultraviolet light source at opposite sides thereof. The implement may be constructed to facilitate any number of manually directed objectives, from opening a door, pushing a grocery cart, turning on a light switch, or operating a computer, for example. So long as the implement is prone to manipulation, particularly by multiple users, it may serve a self-sterilizing germicidal role in preventing the propagation of pathogens from one user to the next by way of the noted contact surface. Among other aspects, this is facilitated by the unique architecture of the implement and the relationship of the ultraviolet light source positioning relative the contact surface to be treated.

### PRIORITY CLAIM/CROSS REFERENCE TO RELATED APPLICATION(S)

This Patent Document claims priority under 35 U.S.C. § 119 to U.S. Provisional App. Ser. No. 63/100,324, filed Mar. 9, 2020, and entitled, “Ultra-Violet Light Sterilized Doorknob”, which is incorporated herein by reference in its entirety.

### BACKGROUND

Utilizing generally short-wavelength ultra-violet light for purposes of sterilization has been accepted practice for many decades. Microorganisms, including bacteria, viruses, molds and other pathogens may be exposed to ultra-violet light in order to kill such pathogens. So, for example, ultra-violet (UV) light devices may be incorporated into water or air purification systems as part of an overall management effort to make sure large-scale public air and water supplies are kept largely free of such live pathogens. By way of example, UV light water purification is generally considered more effective than boiling water where it comes to neutralizing pathogens from a water supply.

Generally speaking, depending on intensity, germicidal UV light that is considered effective against such pathogens while at the same time not considered to present undue risk when it comes to human exposure may be found in a range of between about 200 and about 300 nm. This range of UV is generally considered effective against pathogens in terms of either directly killing such organisms or at least being sufficient to result in prohibiting replication of the organism. In either case, a pathogen exposed to such a range of UV light for several seconds is considered neutralized. In cases where such levels of UV light are at risk of coming into contact with human skin during the sterilization process, the risk is viewed as similar to that of exposure to sunlight, for example, in terms of risking a sunburn. Regardless, the effectiveness of UV treatment may be considered greater than 90-99% in terms of the percentage of pathogens which may be rendered neutralized, of course, depending on the particular protocol and precise circumstances.

Air or water sterilization are not the only sterilization uses for UV light. Indeed, more direct and discrete uses of ultra-violet (UV) light may be employed to ensure that specific items are kept free of live pathogens. For example, medical or consumer sanitation of specific, discrete items may be employed where a UV light is made available for directing at items meant for human contact and use or consumption.

Handheld UV lamps and wands are often utilized to direct UV light at specific items for which sterilization is sought. With the advent of commercially available UV LED's, this practice has grown exponentially. From small solar cells that might be used to sterilize medical equipment in a third world mobile hospital to handheld UV wands in any number of manual packaging sites for consumer goods, the availability of UV sterilization has become widespread.

Unfortunately, even though cost is no longer a substantial obstacle, expanding the availability of UV sterilization beyond such isolated or controlled settings to more public use settings remains largely impractical. For example, while it may be desirous to have public doorways, grocery carts or other mass contacted surfaces available to regular UV sterilization, this remains a challenge. That is, unlike the isolated medical tent or water treatment facility, sterilizing a publicly used door handle requires repeated exposure of the handle to a UV wand or light which may not be practical. Once more, the lack of a controlled environment means that UV treatment issues which are present, even in a controlled environment, may now be amplified. For example, any UV treatment is limited by intervening, shadowing, debris or any number of interference issues. That is, to the extent that any such issues emerge between the UV light source and the surface to be treated, the treatment may be compromised. This is true in the controlled environment and is certainly amplified in public areas where control over such interferences may be near impossible.

Public areas with common contact surface locations such as the noted door handles remain subject to a variety of UV treatment limitations. Apart from the noted potential for light interference, distance variation may affect maintaining stability of light intensity for treatment. Furthermore, given the public nature of such locations, risk of unintended human exposure to the UV treatment remains. Thus, as a practical matter, germicidal treatment of such public surfaces remains largely a matter of utilizing conventional cleaning products and human labor at intermittent times with inconsistent levels of effectiveness.

### SUMMARY

A self-sterilizing implement is disclosed that is meant for manual manipulation by a user. The implement includes a substrate that is either substantially transparent or substantially translucent to ultraviolet light. An ultraviolet light source may be housed within the substrate whereas the substrate includes an outer surface that is configured for interfacing contact with the user during the manual manipulation.

### BRIEF DESCRIPTION OF THE DRAWINGS

Implementations of various structure and techniques will hereafter be described with reference to the accompanying drawings. It should be understood, however, that these drawings are illustrative and not meant to limit the scope of claimed embodiments.

FIG. 1 is a front view of an embodiment of a self-sterilizing implement in the form of a door handle.

FIG. 2 is a cross sectional view of the door handle taken from 2-2 of FIG. 1 revealing an internal ultraviolet light source.

FIG. 3 is a perspective view of a common area hallway accommodating a host of doors with door handles as illustrated in FIG. 1.

FIG. 4 is a side cross-sectional view of a door accommodating an alternate embodiment of a door handle as a self-sterilizing implement.

FIG. 5 is a flow-chart summarizing an embodiment of utilizing a self-sterilizing implement to substantially eliminate the passing of live pathogen from one user to another via the implement.

### DETAILED DESCRIPTION

In the following description, numerous details are set forth to provide an understanding of the present disclosure. However, it will be understood by those skilled in the art that the embodiments described may be practiced without these particular details. Further, numerous variations or modifications may be employed, which remain contemplated by the embodiments as specifically described.

Embodiments are described with reference to particular self-sterilizing implements that are meant for manual manipulation by a user. Different types of door handles are most notably illustrated. However, a variety of other implements may be utilized which take advantage of the architecture and principles detailed herein. For example, keyboards, smartphones, computers, light switches, remote controls, gaming interfaces, screens, buttons, steering wheels, handles, bars, buckles and any other numerous types of implements meant for manual interfacing may benefit from these concepts. So long as the implement itself includes a substrate with an outer surface for interfacing contact with the user during use of the implement while at the same time accommodating an ultraviolet light source therein, appreciable benefit may be realized.

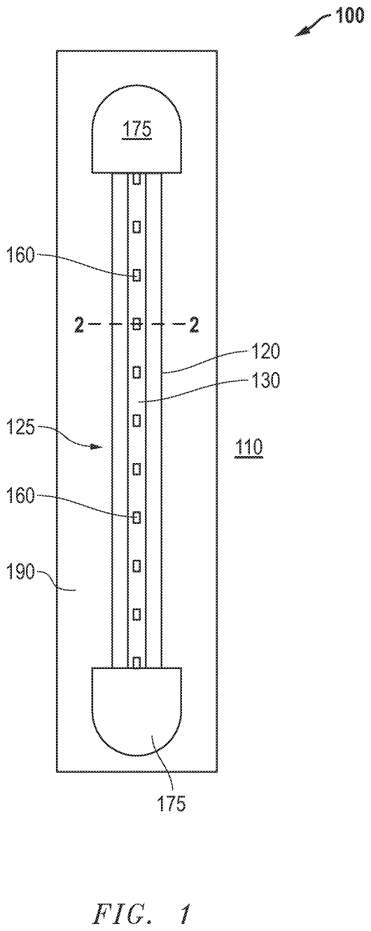

Referring now to FIG. 1, a front view of an embodiment of a self-sterilizing implement 100 in the form of a door handle assembly is shown. The handle assembly 100 includes a bar-shaped structure or lever 125 with an outer surface 120 for contacting by a user in opening a door. In this sense, the assembly 100 is of an architecture for facilitating a door in opening or closing as with a typical door handle. In the depicted embodiment, the lever 125 is depicted for pushing a door open. For example, in the environment of a sick room or operating room, such a handle lever 125 may be pushed from either side for entry into or exiting from the room. In one embodiment, pushing in either direction at the lever 125 may extend a cover over a door latch if present to allow door opening.

For other embodiments discussed below, the assembly 100 of FIG. 1 is also configured for being pulled or grabbed at the surface 120 any number of times by any number of different people throughout any given period of time. Regardless, the surface 120 is likely to come into contact with a variety of different pathogens throughout this given period of time. This means that, in absence of some form of germicidal intervention, the assembly 100 might serve as an excellent medium for the transfer and spread of live pathogen from one door handle user to the next and to the next and so on. Fortunately, embodiments herein are directed at unique germicidal architecture and techniques achieved through such an implement as the depicted door handle assembly 100.

Continuing with reference to FIG. 1, the handle assembly 100 is equipped with an ultraviolet light source 130. More specifically, in the embodiment shown, the light source is supplied in the form of an ultraviolet (UV), light emitting diode (LED) array 130 consisting of a variety of discrete UV LED's 160 for emitting UV C light as detailed further below. As also detailed further below, it is understood that UV light, including from LED's 160, may serve as an effective germicidal agent. However, in the present embodiments, the UV light from the LED's 160 is uniquely supplied relative the noted medium of the contact surface 120 where there is the potential for pathogen presence.

For the embodiment of FIG. 1 and others herein, the ultraviolet light source 130 is found housed within the substrate or structure of the implement lever 125. This means that the contact surface 120 which is prone to pathogen exposure is oriented opposite the location of the light source 130 and that the source 130 itself is kept at a shielded location. More specifically, the substrate of the lever structure 125 itself may protect the light source 130 from a variety of different types of interference that might otherwise mitigate its effectiveness as a germicide. For example, the opportunity for dust, fog or other debris to cast a shadow between the source 130 and the contact surface 120 is eliminated due to the embedded and shielded nature of the source 130 within the lever 125.

In order to ensure the effectiveness of the source 130 as a germicidal agent in reaching the contact surface 120, the substrate material of the lever 125 may be of a substantially transparent material such as quartz. In an alternate embodiment, the substrate material is substantially translucent with a mix of coloring or scattering agent intentionally incorporated therein, for example, along with a base quartz material. In either circumstance, the effectiveness of ultraviolet light in reaching the contact surface 120 for germicidal treatment is substantially unhindered. In an embodiment where a scattering agent is employed, the distribution of the light may be enhanced and/or further propagated toward the surface 120.

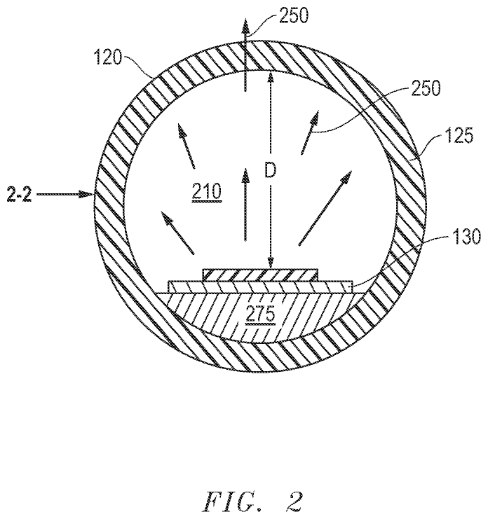

With added reference to FIG. 2, the substrate of the quartz handle structure 125 is provided in tubular form as detailed further below. This means that apart from the substrate material of the structure 125, an airspace 210 may be found within the tubular structure 125 between the outer surface 120 and the light source 130. However, the isolated nature of the airspace 210, like the discrete LED's 160, of the source 130, maintains a level of protection from interference or shadowing between the surface 120 and the light source 130, 160.

In terms of assembly particulars, the source 130 and/or LED's 160 may be configured to emit UV-C light (e.g. see 250 of FIG. 2). UV-C light is ultraviolet light found in the range of between about 100 and 280 nm and may be particularly effective as a germicidal agent without undesired effects, for example the potential to inflict a sunburn at a user's skin.

As discussed above, the surrounding housing structure of the handle lever 125 is a substrate with an outer surface 120 susceptible to collection of pathogens due to user manual manipulation. Thus, the ability of the light 250 to reach the surface 120 as illustrated in FIG. 2 is noteworthy. Along these lines, the substrate may be of calcium fluoride, fused silica or a quartz-based material. For example, quartz itself is substantially transparent to such UV light, depending on the degree of purity. Alternatively, quartz with coloring or disbursing agent mixed therein may be utilized, for example, to promote light scattering while remaining substantially translucent for allowing the light to reach the surface 120. In either case, such quartz-based structures are commonly available in tubular form. Thus, whether employed to form a handle lever 125 for a door, or for a grocery cart as detailed below, manufacturability and supply issues need not be of significant concern.

UV-C replacement bulbs, LED strips or arrays 130, and even discrete LED's 160 are also readily available on the commercial market. Thus, the handle assembly 100 as illustrated in FIG. 1 may be readily constructed. In the specific embodiment shown, a base 190 accommodates the components detailed above by way of end caps 175. However, other handle configurations may be employed with power supplied to the array 130 or LED's 160 as detailed below, perhaps 2-5 volts.

Referring now to FIG. 2, a cross sectional view of the door handle/lever 125 is shown taken from 2-2 of FIG. 1. In this view the internal ultraviolet light source 130 is shown in strip form accommodating a plurality of UV LED's 160 (one being shown in cross-section). Due to the tubular nature of the lever 125 structure, an adhesive support base 275 is shown to facilitate coupling of the strip 130 to the interior surface of the tubular lever 125. Of course, depending on dimensions, the strip 130 may be more directly adhered to this inner surface without need of any substantial interfacing base 275 as shown. Furthermore, in another embodiment, the strip 130 itself may be of a more rigid nature for suspending the discrete LED's 160 from a more central tubular location, supported by the end caps 175 instead of directly to the tubular lever structure 125. In this way, light 250 may be emitted effectively from both sides of each LED for reaching the entire circumference of the surface 120 at hand.

In terms of dimensions, the ultraviolet light 250 is meant to traverse a given, potentially variable, distance (D) in order to reach the noted outer surface 120 where germicidal behavior may take place as described above. Recalling that the dimensions involved here are in the field of a manual lever or implement, regardless of the particular embodiment (e.g. door handle or otherwise). Thus, with the average human hand being well below about 7.5 inches from base to fingertip, the distance (D) is a matter of inches itself, certainly less than 7 inches. Once more, the implement may be less of a lever/handle 125 configuration for grabbing and more of a button, interface screen or other surface meant for merely touching or pressing. Thus, the distance (D) may traverse a non-tubular, potentially smaller, region in reaching the outer surface 120. Therefore, it may be expected for a majority of embodiments to include a distance (D) that is less than about 7 inches.

With the limited distance (D) in mind, it is worth noting that, as with other types of commercially available light sources, the ultraviolet light 250 is quantified, in terms of intensity, based on a distance of a meter (i.e. 39.4 inches). So, for example, where the LED's 160 of the strip 130 of FIG. 2 are rated at 2-8K μWs/cm², they are not only more than sufficient to serve as a germicidal, the decrease in intensity over the less than 7 inches of distance (D) would be negligible, meaning that the light 250 would remain a more than sufficient germicidal upon reaching the surface 120 at issue.

Continuing with reference to the embodiment of FIG. 2, an airspace 210 is depicted within the handle structure 125 along with the LED components. However, the contact surface 120 and underlying structure 125 still serve as a substrate for housing these components. Thus, the light 250 traverses isolated air, substantially free of debris, shadowing particulate, fog or other intervening materials that might affect the quality of the light 250 reaching the surface 120. Further, the substrate structure 125 leading to the surface 120 is either substantially transparent or translucent itself, such as quartz, further minimizing any deleterious effect on the ability of the light to reach the contact surface 120. Indeed, as discussed above, even the intensity of the light is unlikely to be diminished in any perceptible manner due to the close proximity of the light 250 to the surface 120.

For the embodiments described hereinabove, focus is so far drawn to the use of UV light 250 to serve as a germicide for a contact surface 120 and the particular architecture employed, namely with the light source 130, 160 being housed within or below the surface 120. However, a variety of other aspects are to be considered. For example, a variety of different triggering and timing features may be employed with such assemblies. This may include triggering UV light 250 to be emitted based on a sensed push on the door handle assembly 100 of FIG. 1, such as through incorporation of a piezo electric sensor within one of the end caps 175 or by way of a magnetic sensor at a door jam (see FIG. 1). Regardless, beyond triggering, there may be an intentionally delayed triggering, timing of the light 250 emissions and a variety of other considerations, perhaps tailored to the particular nature of the implement and the environment at hand. Such aspects are considered further below with respect to additionally detailed embodiments.

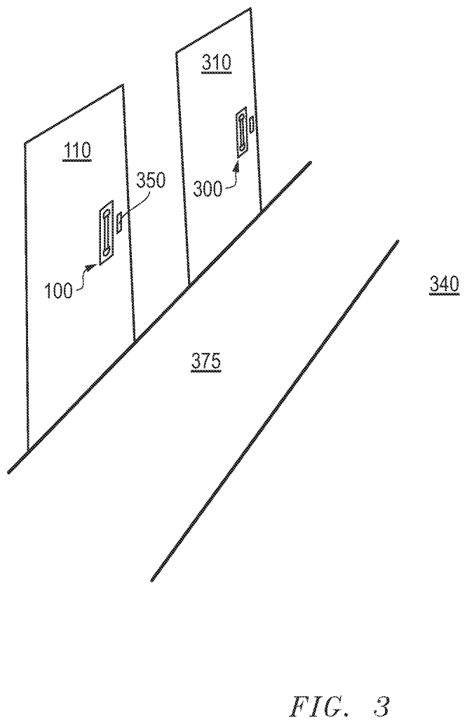

Referring now to FIG. 3, a perspective view of a common area hallway 375 is shown accommodating a host of doors 110, 310 with door handles 100, 300 as illustrated in FIG. 1. This is meant to illustrate a common area such as the hallway 375 in a public environment 340 of a hotel, shopping center or office building where a host of different people are likely to pass and periodically push or grab onto such handles 100, 300. As a result, anyone making contact with the handle 100, 300 is prone to introduce pathogens to the contact surface 120 as illustrated in FIG. 1.

In the embodiment of FIG. 3, a power source 350 is illustrated at the doors 110, 310. Continuing with the example embodiment of a hotel, the source may be provided as part of a standard card reader package that is electronically linked to various handle and latch components of a hotel door 110, 310. With added reference to FIGS. 1 and 2, in this case, the powered linkage is also coupled to a UV light source 130, 160 to supply UV light 250 to a handle contact surface 120 as described above.

For ease of illustration, in keeping with the hotel example, a visitor, guest, cleaning personnel or any other user may grab a handle 100, 300 to gain room entry. With added reference to FIGS. 1 and 2, by making contact with the surface 120 during the grabbing risk of pathogen exposure is presented to the surface 120. Thus, in order to kill the pathogen prior to grabbing by the next user, the UV light 250 is activated. In one embodiment, this takes place by a trigger sense mechanism which senses the pulling or a pushing as the case may be. As discussed above, the mechanism may include a conventional piezoelectric actuator incorporated into the handle 100, 300, perhaps at one or both end caps 175, with relay to the depicted power source 350. Of course, various other types of conventional triggers are available which may be utilized.

In another embodiment, processing means may be incorporated into the power source package 350. Thus, the actual turning on and emitting of the UV light 250 may be delayed, by a predetermined period after sensing of the push or pull, may be 2-5 seconds, or until the pull is no longer sensed. Thus, direct UV exposure to the user may be avoided with the light 250 focused solely at the contact surface 120 and potential pathogens left behind. In this way, risk of UV harm to the user may be avoided. Alternatively, where the UV light 250 is viewed as having such a minimal intensity and exposure time that such risk is a non-issue, the processor may be programmed to immediately direct UV light 250 emission upon pushing or pulling. As a result, for the minimal period that the user grabs onto the handle 100, 300, some level of germicide may be applied directly to the current user's hand in addition to the contact surface 120 for the benefit of the current user as well as a future user. Along these lines, a proximity switch may even be utilized to trigger UV light 250 emission a moment before actual user contact is made with the surface 120. Further, regardless of when the light 250 begins to be emitted, there is likely a set predetermined period of time, for example 10-15 seconds, after which the light 250 is stopped. The predetermined emission time may be tied to factors such as light intensity, the particular environment, user safety, power savings and so forth.

The embodiment of FIG. 3 details various examples of a layout for the system, for example, with a package 350 that accommodates a processor and power source near the handle 100, 300. However, this is only exemplary. For example, a power may be hard wired through a door hinge without any separately required package. This may be advantageous in environments where an available package site is not already available or simply to ensure continuous power availability without requiring battery change outs. By the same token, additional visible light may be used for purposes apart from serving as a germicide. For example, continuing with reference to the example of a public hallway 375, a light sensor incorporated with the depicted package 350 or elsewhere, may cooperate with the processor to trigger emission of visible light whenever a light of the hallway 375 falls below a predetermined level. That is, it may be helpful to light the hallway 375 with UV door handles 100, 300 for the public in the area, for example, when a fire or other emergency has cut off regular lighting to the hallway 375.

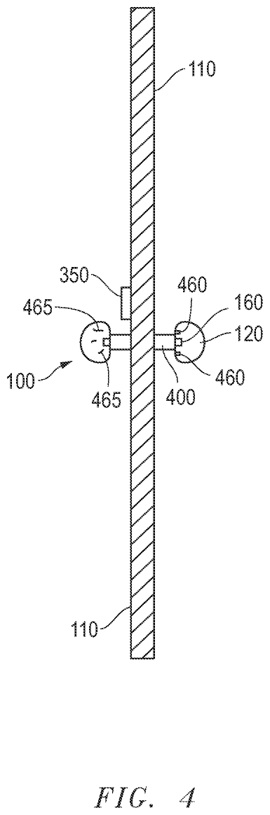

Referring now to FIG. 4, a side cross-sectional view of a door 110 accommodating an alternate embodiment of a door handle 100 as a self-sterilizing implement is shown. In this embodiment, the same principles above are employed. However, the architecture of the handle 100 is different. Specifically, rather than a tubular variety, the handle 100 is a more conventional residential knob-shaped variety. This configuration may be more suitable for accommodating fiber optic UV sources 460 that emerge laterally from a door pull 400 to emit the UV light 465. This may provide a suitable battery location within the pull 400. Further, instead of employing a conventionally available tubular form with a large volume of airspace 210 the substrate structure of the quartz handle may be of a more solid, specially configured form (see FIG. 2). Minimal, tailored vias to accommodate fiber optics directed at the surface 120 may be the extent of potential for airspace. In this way, accommodating fiber optic UV sources 460, or even a visible LED light source, may be done in a more tailored and directed manner with tighter constraints on airspace and LED emission direction. Regardless, any combination of visible or non-visible UV fiber optic and/or UV LED mix may be employed for the depicted embodiment.

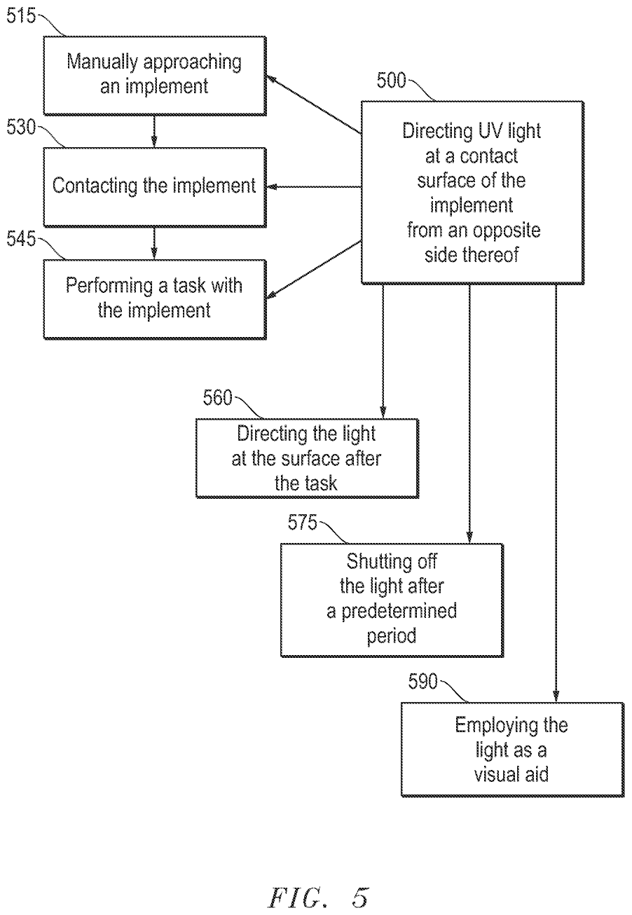

Referring now to FIG. 5, a flow-chart is depicted summarizing an embodiment of utilizing a self-sterilizing implement to substantially eliminate the passing of live pathogen from one user to another via the implement. Specifically, as indicated at 500, UV light may be directed at a contact surface of an implement from within or from an opposite side of the contact surface. This may occur as the implement is approached 515, during the contacting 530, as a task is performed with the implement 545 or for a set period after the performing of the task 560. Regardless, the UV light would generally be shut off after a predetermined period (see 575). Furthermore, the UV light may even be employed for other visual aid purposes as indicated at 590.

Embodiments described hereinabove include architecture and techniques that may be employed for implements to achieve reliably consistent levels of germicidal effectiveness. Limitations in terms of light interference and inconsistent intensity are substantially eliminated. Once more, the risk of undesired human exposure to ultraviolet light is also substantially eliminated. As a result, reliance on human labor, chemicals and other potentially harmful and inconsistent modes of cleaning publicly utilized implements may be avoided while still attaining beneficial germicidal results.

The preceding description has been presented with reference to presently preferred embodiments. Persons skilled in the art and technology to which these embodiments pertain will appreciate that alterations and changes in the described structures and methods of operation may be practiced without meaningfully departing from the principle, and scope of these embodiments. For example, readily replaceable or disposable configurations may be employed such as in the form of sleeve assemblies for grocery cart handles. Specially configured light switch plates and/or toggles for a dwelling may also employ concepts detailed herein. Such embodiments may be provided in packaging and kits that allow for simple switch out with already present conventional switch assemblies. Such novel UV switch assemblies may include specialized circuitry that supports UV triggering, timing and other parameters detailed hereinabove, while also being suitable for coupling to already present house wiring. Furthermore, the foregoing description should not be read as pertaining only to the precise structures described and shown in the accompanying drawings, but rather should be read as consistent with and as support for the following claims, which are to have their fullest and fairest scope.

I claim:

1. A self-sterilizing implement for manual manipulation by a user, the implement comprising:
a substrate that is one of substantially transparent and substantially translucent to ultraviolet light;
an ultraviolet light source adjacent a first side of the substrate; and
an outer surface of the substrate at a second side thereof, opposite the first side and for interfacing contact with the user during the manual manipulation.

2. The self-sterilizing implement of claim 1 wherein the substrate is of a structural material selected from a group consisting of calcium fluoride, fused silica and a quartz-based material.

3. The self-sterilizing implement of claim 2 wherein the quartz-based material is a tubular structure for housing the ultraviolet light source therein.

4. The self-sterilizing implement of claim 1 wherein the substrate is of a knob configuration for housing the ultraviolet light source therein.

5. The self-sterilizing implement of claim 1 wherein the ultraviolet light source includes one of an LED and a fiber optic light source.

6. The self-sterilizing implement of claim 1 wherein the implement is configured as one of a button, screen, switch, wheel, buckle, bar, handle and lever.

7. The self-sterilizing implement of claim 6 wherein the button is a button for one of a keyboard and a remote, the screen is a screen for one of a smartphone, a computer and a gaming interface, the wheel is a steering wheel, and the switch is a light switch.

8. The self-sterilizing implement of claim 6 wherein the one of the lever, bar and handle is constructed as a replaceable, tubular sleeve for a grocery cart.

9. A self-sterilizing system, comprising:
an implement having a structure that is one of substantially transparent and substantially translucent with an outer surface for manual manipulation by multiple users, the implement having an adjacent ultraviolet light source at a location opposite the outer surface;
a power source electrically coupled to the ultraviolet light source; and
a processor coupled to the power source to direct powering of the ultraviolet light source.

10. The self-sterilizing system of claim 9 wherein the multiple users are supplied by an environment selected from a hotel, a shopping center and an office building.

11. The self-sterilizing system of claim 9 wherein the system is configured for use with a door with the implement in the form of a door handle.

12. The self-sterilizing system of claim 11 further comprising an electronics package secured at the door to accommodate one of the power source and the processor.

13. The self-sterilizing system of claim 11 wherein the power source is an external power source hard wired through a hinge at the door to support powering of the ultraviolet light source at the handle.

14. The self-sterilizing system of claim 9 wherein the system is configured for use with a light switch assembly of a dwelling with the implement in the form of one of a toggle and a switch plate.

15. The self-sterilizing system of claim 14 wherein the implement and the processor are configured as a replaceable package for coupling to preplaced power source wiring of the dwelling.

16. A method of sterilizing an implement for manual manipulation by a user, the method comprising directing ultraviolet light at a contact surface of the implement from an opposite side thereof.

17. The method of claim 16 further comprising manually contacting the contact surface to trigger the directing of the ultraviolet light.

18. The method of claim 17 wherein the directing of the ultraviolet light at the contact surface is for a predetermined period after the manually contacting thereof.

19. The method of claim 18 wherein the predetermined period is initiated after a predetermined time delay from the manually contacting of the contact surface.

20. The method of claim 16 further comprising directing visible light from the implement as a visual aid for the user.
